(19) United States
(12) Patent Application Publication
(10) Pub. No.: US 2022/0081387 A1
(43) Pub. Date: Mar. 17, 2022

(54) METHODS FOR PREPARING AMMONIUM TETRATHIOMOLYBDATE

(71) Applicant: Alexion Pharmaceuticals, Inc., Boston, MA (US)

(72) Inventors: Viveca Oltner, Malmo (SE); Niklas Wahlstrom, Malmo (SE)

(73) Assignee: Alexion Pharmaceuticals, Inc., Boston, MA (US)

(21) Appl. No.: 17/421,511

(22) PCT Filed: Jan. 14, 2020
(86) PCT No.: PCT/IB2020/050276

§ 371 Date: Jul. 8, 2021

Related U.S. Application Data
(60) Provisional application No. 62/792,292, filed on Jan. 14, 2019.

(51) Int. Cl. C07C 213/08; C01G 39/00

(52) U.S. Cl. CPC C07C 213/08; C01P 2006/80; C01G 39/006

(57) ABSTRACT

This disclosure relates to crystalline ammonium tetrathiomolybdate having pharmaceutical grade purity and processes for manufacturing crystalline ammonium tetrathiomolybdate. This disclosure also relates to processes for manufacturing bis-choline tetrathiomolybdate having pharmaceutical grade purity.

### CROSS-REFERENCE TO RELATED APPLICATIONS

This application claims the benefit of priority of U.S. Provisional Patent Application No. 62/792,292, filed Jan. 14, 2019, all of which is incorporated by reference in its entirety.

### BACKGROUND OF THE DISCLOSURE

### Field of the Disclosure

This disclosure relates to crystalline ammonium tetrathiomolybdate having pharmaceutical grade purity and processes for manufacturing crystalline ammonium tetrathiomolybdate. This disclosure also relates to processes for manufacturing bis-choline tetrathiomolybdate having pharmaceutical grade purity.

### Description of Related Art

Ammonium tetrathiomolybdate ((NH₄)₂MoS₄; ATTM) is one of the regulatory starting materials for manufacture of bis-choline tetrathiomolybdate (BC-TTM). An example of one reaction to prepare BC-TTM is shown in Scheme 1.

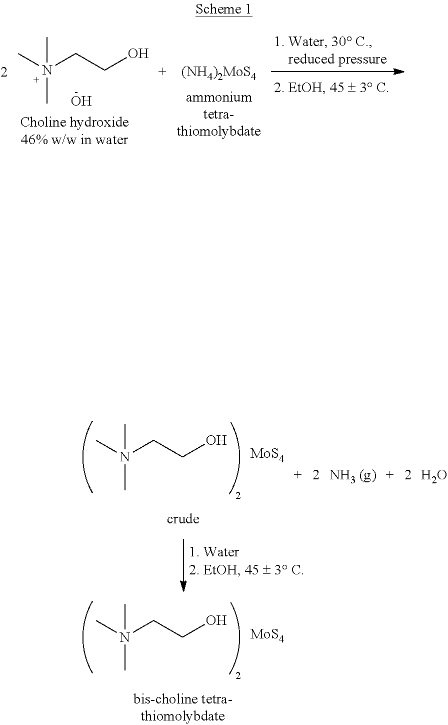

It was determined that the quality of BC-TTM is significantly influenced by the quality of ATTM (i.e., the purity of ATTM).

ATTM is commercially available; however, the commercially available ATTM contains relatively large amounts of impurities. Above all, the impurity profile of commercially available ATTM differs greatly from batch to batch and also between commercial providers. Furthermore, the batch sizes of commercially available ATTM are relatively small, which indicates that the commercial production of ATTM is performed on a lab-scale. The small scale of the current nominally ‘commercial’ production affects the supply and ability to perform the BC-TTM reaction, and potentially can introduce regulatory and safety issues with using many batches of ATTM.

Moreover, the commercial ATTM production uses a molybdate salt (MoO₄²⁻) in reaction with hydrogen sulfide (H₂S) to obtain ATTM:

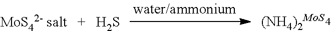

Hydrogen sulfide is a highly poisonous, corrosive, toxic, and flammable gas. Thus, not only does H₂S use limit the production to a lab-scale or to specialized equipment set ups that can handle such a reagent, but its use is highly problematic from health, safety, and environmental perspectives.

Therefore, there remains a need for a process to prepare ATTM in high purity and yield on a large manufacturing scale by minimizing the use and/or emission of the toxic H₂S gas.

### SUMMARY OF THE DISCLOSURE

The disclosure provides efficient processes to obtain ATTM having low impurities. Specifically, the disclosure provides processes for manufacturing crystalline ATTM. Such processes include:
contacting a molybdenum compound selected from ammonium heptamolybdate, ammonium dimolybdate, sodium molybdate, molybdenum oxide, and hydrate thereof with water and ammonia in a reaction vessel under stirring to obtain a molybdenum compound solution;
providing ammonium sulfide to the molybdenum compound solution in an amount corresponding to a S:Mo molar ratio in a range of 4.5:1 to 6.5:1 over a period of time sufficient to obtain a reaction mixture;
maintaining the reaction mixture at a temperature in a range of 35° C. to 55° C. for a reaction time of at least 4 hours to create a slurry;
optionally providing a crystallization solvent to the slurry;
optionally maintaining the slurry at a temperature in a range of 10° C. to 30° C. for at least 2 hours; and
removing liquid from the slurry to obtain crystalline ATTM.

Another aspect of the disclosure provides crystalline ATTM obtained by processes as described herein having pharmaceutical grade purity.

In certain embodiments, the crystalline ATTM obtained by such processes is at least 95%, or at least 96%, or at least 97%, or at least 98%, or at least 99% pure on a weight/weight (w/w) basis.

In certain embodiments, the crystalline ATTM obtained by such processes has less than 5%, or less than 4%, or less than 3%, or less than 2.5%, or about 2% (w/w) of [MoOS₃]²⁻ (TM3) impurity.

Another aspect of the disclosure provides processes for manufacturing bis-choline tetrathiomolybdate, wherein bis-choline tetrathiomolybdate has pharmaceutical grade purity. Such processes include:
contacting an aqueous choline hydroxide solution with a crystalline ammonium tetrathiomolybdate obtained by processes as described herein and water to obtain a reaction mixture;
maintaining the reaction mixture at a temperature in a range of 10° C. to 40° C. for a reaction time sufficient to obtain crude bis-choline tetrathiomolybdate; and
providing ethanol to the reaction mixture at a temperature in a range of 35° C. to 55° C. over a period of time sufficient to obtain bis-choline tetrathiomolybdate.

In certain embodiments, the bis-choline tetrathiomolybdate obtained by such processes has less than 0.5%, or less than 0.25%, or less than 0.2% (w/w) of TM3 impurity.

### BRIEF DESCRIPTION OF THE DRAWINGS

The accompanying drawings are included to provide a further understanding of the methods and materials of the disclosure, and are incorporated in and constitute a part of this specification. The drawings illustrate one or more embodiment(s) of the disclosure and, together with the description, serve to explain the principles and operation of the disclosure.

FIG. 1 illustrates a reactor system suitable for use in the processes of the disclosure.

FIG. 2 illustrates a reactor system suitable for use in the processes of the disclosure.

### DETAILED DESCRIPTION

Before the disclosed methods and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the processes described herein can be configured by the person of ordinary skill in the art to meet the desired need. In general, the disclosed processes provide processes to manufacture, on a large scale, ammonium tetrathiomolybdate in excellent purity and yield. For example, the current processes allow for the manufacture of ATTM on a kilogram-scale. In certain embodiments, the crystalline ATTM is obtained in yield of at least 75%, and often over 80% or 83%, using the processes of the disclosure. Moreover, in certain embodiments, such crystalline ATTM has excellent purity of at least 96% (w/w) pure, and often greater than 98% (w/w) pure, with the most undesired impurity, TM3, at levels of far less than 4% (w/w), for example less than 3%, or less than 2.5%, or even less than 2% (w/w).

Furthermore, the processes of the disclosure, as illustrated in the example chemical reaction below, use ammonium sulfide to obtain ATTM. Therefore, the processes of the disclosure avoid serious health, safety, and environmental concerns associated with the use of hydrogen sulfide. In certain embodiments, the processes of the disclosure are configured to comply with good manufacturing process (cGMP) standard.

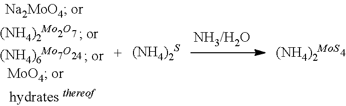

Thus, one aspect of the disclosure provides processes for manufacturing crystalline ATTM. Such processes include contacting a molybdenum compound with water and ammonia in a reaction vessel under stirring to obtain a molybdenum compound solution. Molybdenum compounds suitable for use in the processes of the disclosure include ammonium heptamolybdate, ammonium dimolybdate, sodium molybdate, molybdenum oxide, and hydrates thereof. In certain embodiments, the molybdenum compound of the disclosure is selected from ammonium heptamolybdate, ammonium dimolybdate, sodium molybdate, and hydrate thereof. In certain embodiments, the molybdenum compound of the disclosure is selected from ammonium heptamolybdate, ammonium dimolybdate, and hydrate thereof. In some embodiments of the disclosure, the molybdenum compound is ammonium heptamolybdate or ammonium heptamolybdate tetrahydrate. In some embodiments of the disclosure, the molybdenum compound is ammonium heptamolybdate. In some embodiments of the disclosure, the molybdenum compound is ammonium heptamolybdate tetrahydrate. In some embodiments of the disclosure, the molybdenum compound is ammonium dimolybdate.

In certain embodiments, the ammonia provided to the molybdenum compound is provided in an amount sufficient to ensure minimal release of hydrogen sulfide during the reaction to the gas phase. In certain embodiments, ammonia is provided in an amount sufficient for the molybdenum compound solution to be at about pH 10.

The process of the disclosure includes providing ammonium sulfide to the molybdenum compound solution to obtain a reaction mixture. In certain embodiments, ammonium sulfide may be provided to the molybdenum compound solution for a period of time sufficient to obtain a reaction mixture, such as at least 15 minutes, or at least 20 minutes, or at least 30 minutes. Ammonium sulfide may be provided as an aqueous solution. Ammonium sulfide may be at least 10% aqueous solution, or at least 15% aqueous solution, or at least 20% aqueous solution, or about 20 to 48% aqueous solution, or about 20 to 24% aqueous solution, or about 40 to 48% aqueous solution.

The amount of ammonium sulfide suitable for use in the processes should be sufficient to provide good yield and robust process, but also not excessive in order to minimize the amount of toxic hydrogen sulfide that is produced in the process. Thus, in the processes of the disclosure, the amount of ammonium sulfide that is provided to the molybdenum compound solution is an amount that corresponds to a S:Mo molar ratio in a range of 4.5:1 to 6.5:1.

In certain embodiments of the processes of the disclosure, ammonium sulfide is provided to the molybdenum compound solution in an amount corresponding to a S:Mo molar ratio in a range of 4.5:1 to 6.2:1; or 4.5:1 to 6:1; or 4.5:1 to 5.8:1; or 4.5:1 to 5.5:1; or 4.5:1 to 5.3:1; or 4.5:1 to 5:1; or 4.8:1 to 6.5:1; or 4.8:1 to 6.2:1; or 4.8:1 to 6:1; or 4.8:1 to 5.8:1; or 4.8:1 to 5.5:1; or 4.8:1 to 5.3:1; or 4.8:1 to 5:1; or 5:1 to 6.5:1; or 5:1 to 6.2:1; or 5:1 to 6:1; or 5:1 to 5.8:1; or 5:1 to 5.5:1; or 5:1 to 5.3:1; or 5.2:1 to 6.5:1; or 5.2:1 to 6.2:1; or 5.2:1 to 6:1; or 5.2:1 to 5.8:1; or 5.2:1 to 5.5:1; or 5.5:1 to 6.5:1; or 5.5:1 to 6.2:1; or 5.5:1 to 6:1; or 5.5:1 to 5.8:1; or 5.3:1 to 5.7:1; or 5.4:1 to 5.6:1; or 5.45:1 to 5.55:1. In certain embodiments of the processes of the disclosure, ammonium sulfide is provided to the molybdenum compound solution in an amount corresponding to a S:Mo molar ratio of about 5.5:1; or about 6:1; about 5:1; about 4.5:1.

The reaction time should be sufficient to provide good yield over a reasonable period of time while minimizing the undesirable impurities. Thus, the reaction mixture is maintained at a temperature in a range of 35° C. to 55° C. In certain embodiments of the processes of the disclosure, the reaction mixture is maintained at a temperature in a range of 40° C. to 55° C.; or 45° C. to 55° C.; or 50° C. to 55° C.; or 35° C. to 50° C.; or 40° C. to 50° C.; or 45° C. to 50° C.; or 35° C. to 45° C.; or 40° C. to 45° C.; or 45° C. to 50° C.; or 37° C. to 53° C.; or 38° C. to 52° C.; or 41° C. to 49° C.; or 42° C. to 48° C.; or 43° C. to 47° C.; or 44° C. to 46° C. In certain embodiments of the processes of the disclosure, the reaction mixture is maintained at a temperature of about 45° C.

The reaction mixture is maintained for a reaction time of at least 4 hours. In certain embodiments of the processes of the disclosure, the reaction time is at least 4.5 hours; or at least 5 hours; or in a range of 4 hours to 8 hours; or in a range of 4 hours to 7 hours; or in a range of 4 hours to 6 hours; or in a range of 4 hours to 5 hours; or in a range of 4.5 hours to 8 hours; or in a range of 4.5 hours to 7 hours; or in a range of 4.5 hours to 6 hours; or in a range of 4.5 hours to 5 hours; or in a range of 5 hours to 8 hours; or in a range of 5 hours to 7 hours; or in a range of 5 hours to 6 hours; or in a range of 5.5 hours to 6 hours; or in a range of 4.5 hours to 5.5 hours; or in a range of 4.6 hours to 5.4 hours; or in a range of 4.7 hours to 5.3 hours; or in a range of 4.8 hours to 5.2 hours; or in a range of 4.9 hours to 5.1 hours. In certain embodiments of the processes of the disclosure, the reaction time is about 5 hours. At the end of the reaction time, a slurry of ATTM is formed.

In the processes of the disclosure, a crystallization solvent is optionally provided to the slurry to increase yield from the original reaction slurry. Suitable crystallization solvents are known in the art and may be selected from alcohol (.e.g, methanol, ethanol, etc.), ethylene glycol, tetrahydrofuran, diethyl ether, water, and mixtures thereof. In certain embodiments, the crystallization solvent is ethanol. Ethanol may be provided in an amount of about 30 to 60% by volume; or about 30 to 50 vol %; or about 30 to 40 vol %; or about 30 to 35 vol %; or about 35 to 40 vol %; or about 35 to 45 vol %; or about 33 to 37 vol %; or about 34 to 36 vol %.

In certain embodiments, the crystallization solvent is provided for at least 15 minutes; or for at least 20 minutes; or for at least 25 minutes; or over a time in the range of 15 minutes to 30 minutes; or 15 minutes to 25 minutes; or 15 minutes to 20 minutes; or 20 minutes to 30 minutes; or 20 minutes to 25 minutes; or 18 minutes to 22 minutes; or for about 20 minutes. The crystallization solvent is also provided at a temperature in a range of 35° C. to 55° C. In certain embodiments, the crystallization solvent is provided for at a temperature in a range of 40° C. to 55° C.; or 45° C. to 55° C.; or 50° C. to 55° C.; or 35° C. to 50° C.; or 40° C. to 50° C.; or 45° C. to 50° C.; or 35° C. to 45° C.; or 40° C. to 45° C.; or 45° C. to 50° C.; or 37° C. to 53° C.; or 38° C. to 52° C.; or 41° C. to 49° C.; or 42° C. to 48° C.; or 43° C. to 47° C.; or 44° C. to 46° C. In certain embodiments, the crystallization solvent is provided at a temperature of about 45° C.

The slurry as described herein is then optionally cooled to a temperature in a range of 10° C. to 30° C. The cooling may be gradual, in certain embodiments, for example at a cooling rate of 0.1-2° C./minute; or 0.2-2° C./minute; or 0.1-1° C./minute; or 0.2-1° C./minute; or about 0.6° C./minute; or about 0.5° C./min. In certain embodiments, gradual cooling is over a period of at least 30 minutes; or at least 45 minutes; or at least 1 hour; or between 30 minutes and 1 hour.

The slurry is optionally maintained, optionally with stirring, at the temperature in a range of 10° C. to 30° C. for at least 2 hours. In certain embodiments, the slurry is maintained, optionally with stirring, at a temperature in a range of 10° C. to 30° C. for at least 3 hours; or at least 5 hours; or at least 6 hours; or at least 7 hours; or at least 8 hours; or at least 9 hours; or at least 10 hours; or at least 11 hours; or at least 12 hours; or for a time in the range of 8 hours to 16 hours; or 10 hours to 16 hours; or 8 hours to 14 hours; or 10 hours to 14 hours; or 8 hours to 12 hours; or 10 hours to 12 hours. In certain embodiments, the slurry is maintained, optionally with stirring, at a temperature in a range of 10° C. to 25° C.; or 10° C. to 20° C.; or 10° C. to 15° C.; or 15° C. to 30° C.; or 15° C. to 25° C.; or 15° C. to 20° C.; or 20° C. to 30° C.; or 18° C. to 22° C.; or 19° C. to 21° C.; or at a temperature of about 15° C.; or about 20° C.; or about 25° C.

In the processes of the disclosure crystalline ATTM is obtained by removing the liquid (also labeled “mother liquid” or “mother liquor” herein). Removing the liquid may be performed by suitable methods known in the art. In certain embodiments, the liquid is removed by filtration, such as by nutsche filter, Buechner filter, sintered glass filter, paper filter, or similar filters. In certain embodiments, the liquid is removed by centrifugation.

The process of the disclosure, in certain embodiments, further comprises washing the crystalline ATTM with at least one of ethanol, water, or ethanol:water mixture. In certain embodiments, the process further comprises washing the crystalline ATTM with a 2:1 ethanol:water. In certain embodiments, the process further comprises washing the crystalline ATTM with a 2:1 ethanol:water, followed by washing with ethanol. In certain embodiments, the process further comprises washing the crystalline ATTM with ethanol.

The process of the disclosure, in certain embodiments, further comprises drying the crystalline ATTM at about 25° C. under reduced pressure (e.g. in a vacuum oven).

The process of the disclosure, in certain embodiments, further comprises storing the crystalline ATTM under argon. In certain embodiments, storing is at a reduced temperature (e.g., from −20° C. to 0° C.; or about −15° C.; of about −18° C.; or about −20° C.).

The process of the disclosure also produces hydrogen sulfide byproduct. In certain embodiments, hydrogen sulfide obtained as a byproduct is vented into a second reaction vessel comprising a sodium hypochlorite or hydrogen peroxide solution. The sodium hypochlorite or hydrogen peroxide solution is present in the second reaction vessel in an amount sufficient to quench hydrogen sulfide. In certain embodiments, the sodium hypochlorite or hydrogen peroxide solution is present in an amount of at least 50 mole equivalents, or at least 60 mole equivalents, or at least 61 mole equivalents, or at least 70 mole equivalents. In certain embodiments, the second reaction vessel comprises hydrogen peroxide solution. Hydrogen peroxide may be provided as an aqueous solution, such as at least 20% aqueous solution, or at least 30% aqueous solution, at least 35% aqueous solution, or about 30 to 40% aqueous solution, or about 35% aqueous solution.

The processes of the disclosure are suitable for large scale manufacture. Thus, in certain embodiments, the processes allow for kilogram-scale reaction. For example, the molybdenum compound is provided in an amount of at least 1 kg; or at least 2 kg; or at least 5 kg; or at least 10 kg.

The crystalline ATTM obtained by the processes as described herein has pharmaceutical grade purity. For example, in certain embodiments, crystalline ATTM is at least 88%, or at least 89%, or at least 90%, or at least 91%, or at least 92%, or at least 93%, or at least 94%, or at least 95%, or at least 96%, or at least 97%, or at least 98%, or at least 99%, or at least 100% (w/w) pure.

Crystalline ATTM obtained by the processes as described herein has low levels of TM3 impurity. In certain embodiments, the crystalline ATTM comprises less than 9%, or less than 8%, or less than 7%, or less than 6%, or less than 5%, or less than 4%, or less than 3%, or less than 2.5%, or less than 2%, or less than 1%, or about 2% (w/w) of TM3 impurity.

Other molybdenum impurities, such as [MoO₄]²⁻ (TM0), [MoO₃S]²⁻ (TM1), [MoO₂S₂]²⁻ (TM2), and [MoOS₃]²⁻ (TM3), are also low.

| [MoO₄]²⁻ | [MoO₃S]²⁻ | [MoO₂S₂]²⁻ | [MoOS₃]²⁻ |
|---|---|---|---|
| TM0 | TM1 | TM2 | TM3 |

In certain embodiments, the crystalline ATTM comprises less than 10%, or less than 9%, or less than 8%, or less than 7%, or less than 6%, or less than 5%, or less than 4%, or less than 3% (w/w) of total molybdenum impurities, wherein the molybdenum impurities are selected from one or more of TM0, TM1, TM2, and TM3.

Polymeric molybdenum impurities, such as Dimer S6 and Dimer S7 shown below, are also low.

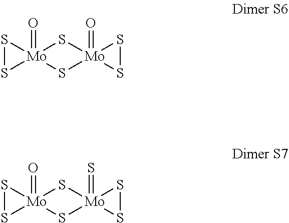

In certain embodiments, the crystalline ATTM comprises no more than 1%, or no more than 0.8%, or no more than 0.6%, or no more than 0.5%, or no more than 0.1%, or no more than 0.05%, or no more than 0.01% (w/w) of polymeric molybdenum impurities.

Another aspect of the disclosure provides processes for manufacturing bis-choline tetrathiomolybdate having pharmaceutical grade purity. Such processes use the crystalline ATTM prepared according to the processes as described herein. As noted above, the purity of the bis-choline tetrathiomolybdate is highly dependent on the purity of ATTM.

In certain embodiments, the processes for manufacturing bis-choline tetrathiomolybdate includes:
contacting an aqueous choline hydroxide solution with crystalline ATTM prepared according to the processes as described herein and water to obtain a reaction mixture;
maintaining the reaction mixture at a temperature in a range of 10° C. to 40° C. for a reaction time sufficient to obtain crude bis-choline tetrathiomolybdate; and
providing ethanol to the reaction mixture at a temperature in a range of 35° C. to 55° C. over a period of time sufficient to obtain bis-choline tetrathiomolybdate.

In certain embodiments, the processes for manufacturing bis-choline tetrathiomolybdate is as provided in the U.S. Pat. No. 7,189,865 B2 (Robert J. Ternansky et al.), incorporated herein by reference in its entirety.

In certain embodiments, the bis-choline tetrathiomolybdate obtained by the processes as described herein has very low levels of TM3 impurity. In certain embodiments, the bis-choline tetrathiomolybdate comprises less than 0.5%, less than 0.25%, less than 0.1%, less than 0.09%, or less than 0.05% (w/w) of TM3 impurity.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

As used in the specification and the appended claims, “alcohol” will be understood to be one or more of a class of organic compounds in which the hydroxy functional group (—OH) is bound to a carbon molecule. Examples include but are not limited to methanol, ethanol, propanol, butanol, pentanol, hexanol, whether straight-chained or branched. Ethanol is of particular interest.

### EXAMPLES

Certain aspects of the disclosure are illustrated further by the following examples, which are not to be construed as limiting the disclosure in scope or spirit to the specific processes and materials described in them.

### Example 1: Process According to an Embodiment of the Disclosure

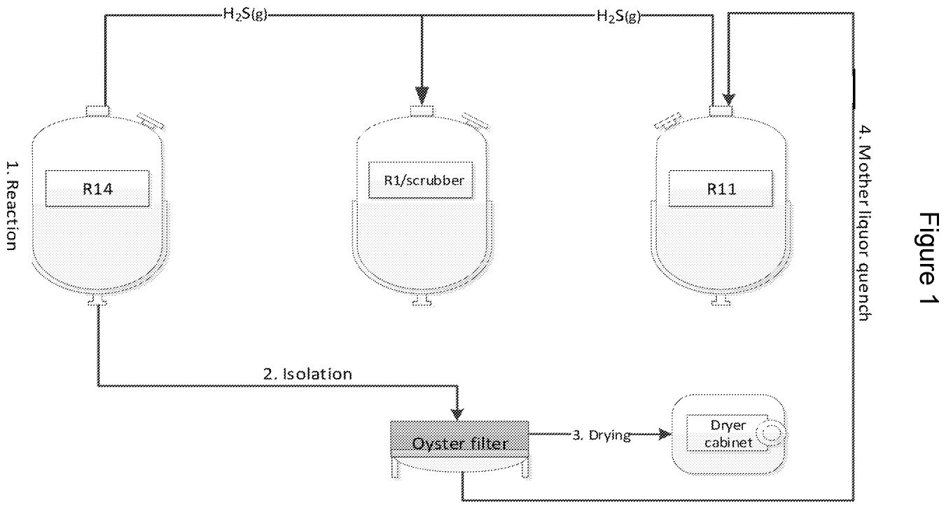

The reactor system used in this process is illustrated in FIG. 1. Briefly, ammonium heptamolybdate (10 kg) was added to reactor R14 followed by water (20 L) and ammonia (5.5 kg, 6 L) and stirred. Ammonium sulfide (97 kg) was added over a period of at least 30 minutes. The temperature was then adjusted to about 45° C. After about 5 hours, ethanol (54 kg, 66 L) was added over a period of at least 20 minutes at about 45° C. After the ethanol addition, the slurry was then gradually cooled to about 20° C. over a period of at least 1 hour and then stirred at least 12 hours at about 20° C. The product was isolated on the nutsche filter and the mother liquids collected in R11. The mother liquid could partly be pumped back to R14 and used to wash down residuals in reactor R14 if needed. The product on the nutsche filter was washed with a mixture of ethanol:water (2:1, 22 L). The mother liquid and wash solution were transferred from R11 to R14. The filter-cake was washed further with ethanol (3×25 L). The wash liquid was transferred from R11 to R14. The product was collected and dried at about 25° C. in a vacuum oven. The drying was done under reduced pressure to a constant weight. The product was thereafter packed under argon and stored in freezer. The mother liquid and wash solutions were reacted with hydrogen peroxide in R14. The quenched solution was also used to wash the nutsche filter and R11.

### Example 2: Comparison with Commercially Available ATTM

ATTM was manufactured according to the processes as described herein, and purity of the crude product was evaluated. The results of three different experiments, 2-1, 2-2, and 2-3, are provided in Table 1. As compared to the commercially available ATTM, the crude products obtained by the processes of the disclosure had lower levels of TM3 and total impurities.

TABLE 1

| | Impurities (% w/w) | | |
|---|---|---|---|
| Example | TM2 | TM3 | Total |
| 2-1 | 0.05 | 4.1 | 4.3 |
| 2-2 | <0.05 | 4.0 | 4.0 |
| 2-3 | 0.07 | 5.9 | 6.2 |
| Commercial ATTM¹ | 0.05 | 8.1 | 8.2 |
| Commercial ATTM¹ | <0.05 | 11.2 | 11.4 |
| Impurity limits | ≤0.5 | ≤10.0 | ≤11.0 |

¹Strem Chemicals, Inc. (Newburyport, MA)

### Example 3: Evaluation of Molybdenum Compounds

Ammonium dimolybdate ((NH₄)₂Mo₂O₇), ammonium heptamolybdate tetrahydrate ((NH₄)₆Mo₇O₂₄ 4H₂O), and sodium molybdate dihydrate (Na₂MoO₄ 2H₂O) were used in the processes of the disclosure. These molybdenum compounds were tested on about 5- to 10-gram scale with the same batch of ammonium sulfide and identical procedures. As provided in Table 2, all three molybdenum compounds resulted in ATTM in 87-89% yields with a purity range of 95.7-96.6% (w/w).

TABLE 2

| | | | | | | | Re- | |
|---|---|---|---|---|---|---|---|---|
| | | | Purity | TM3 | | | action | |
| Ex- | Mo | Yield | (% | (% | | Temp | time | Scale |
| ample | compound | (%) | area) | area) | S:Mo | (° C.) | (h) | (g) |
| 3-1 | (NH₄)₆MO₇O₂₄ | 88.8 | 95.9 | 4.1 | 10:1 | 37 | 20 | 10 |
| 3-2 | 4H₂O | 83 | 97.7 | 2.3 | 5 | 37 | 18 | 5 |
| 3-3 | | 86.8 | 96.7 | 3.3 | 6.5 | 37 | 18 | 5 |
| 3-4 | | 85.8 | 96.7 | 3.3 | 8 | 37 | 18 | 5 |
| 3-5 | | 83.3 | 98.3 | 1.7 | 5 | 37 | 18 | 5 |
| 3-6 | | 32.4 | 98.6 | 1.2 | 5 | 92 | 1.5 | 5 |
| 3-7 | | 80.9 | 96.5 | 3.5 | 5 | 26 | 71 | 5 |
| 3-8 | | 82.6 | 99.1 | 0.9 | 4.5 | 45 | 16 | 5 |
| 3-9 | Na₂MoO₄ | 87.2 | 96.5 | 3.37 | 10:1 | 37 | 18 | 13.7 |
| | 2H₂O | | | | | | | |
| 3-10 | (NH₄)₂Mo₂O₇ | 88.9 | 96.1 | 3.9 | 10:1 | 37 | 18 | 9.6 |
| 3-11 | | 75.1 | 98.8 | 1.2 | 4:1 | 34 | 96 | 5 |
| 3-12 | | 50 | 96 | 4 | 3.8:1 | 60 | ND | 6.9 |

More importantly, ATTM prepared from different molybdenum compounds, when used in the preparation of BC-TTM, also resulted in the product having low impurity levels. As provided in Table 3, BC-TTM was prepared from ATTM, which was prepared in example 3-1, 3-9, and 3-10 above. All three preparations of BC-TTM provided around 78% yield, with excellent analytical characteristics, passing the specification of BC-TTM with good margins.

TABLE 3
Results of BC-TTM preparations from various
ATTM starting materials.

| ATTM starting material | | | | BC-TTM product | | |
|---|---|---|---|---|---|---|
| | Yield | Assay | Impurities | Yield | Assay | Impurities |
| Example | (%) | (% w/w) | (% w/w) | (%) | (% w/w) | (% w/w) |
| 3-1 | 89 | 97.9 | TM3 2.62 | 78 | 99.9 | TM3 0.09 |
| 3-9 | 87 | 101.4 | TM3 0.76 | 78 | 99.1 | TM3 0.30 |
| 3-10 | 89 | 98.1 | TM3 2.28 | 78 | 100.1 | TM3 0.09 |

### Example 4: Evaluation of Solubility and Crystallization Solvent

The ATTM solubility was measured in sample solution (HPLC), methanol, ethanol and purified water. The measurements showed that the ATTM solubility was lowest in alcohols, particularly in ethanol, and higher in water.

After completed reaction, ethanol 35 volume % was added to further precipitate the product. In reactions running at similar scale and about 35° C. the difference in yield with a 35 volume % and 45 volume % ethanol additions was 18%. Running the reaction at about 45° C. omitting crystallization solvent addition reduced the isolated yield to 63%. Addition of 57 volume % ethanol after completed reaction to induce further precipitation gave a product with some late eluting impurities.

### Example 5: Processes According to Some Embodiments of the Disclosure

Two factors which have been found to have high impact on the reaction outcome (yield and purity) are the reaction temperature and the number of equivalents of ammonium sulfide (i.e., S:Mo molar ratio). Several experiments are provided in Table 4. The experiments were performed using the process as provided in Example 1; the reaction time was held constant at 5 hours.

TABLE 4

| | | Temp. | Yield | TM3 | Assay | ATTM |
|---|---|---|---|---|---|---|
| Ex. | S:Mo | (° C.) | (%) | (% w/w) | (% w/w) | (% w/w) |
| 5-1 | 4.5:1 | 35 | 64.7 | 4.50 | 95.6 | 93.0 |
| 5-2 | 6:1 | 35 | 84.2 | 2.57 | 97.5 | 96.0 |
| 5-3 | 4.5:1 | 50 | 76.1 | 1.01 | 98.9 | 98.4 |
| 5-4 | 6:1 | 50 | 90.5 | 0.44 | 98.8 | 99.3 |
| 5-5 | 5.25:1 | 42.5 | 83.4 | 1.35 | 98.9 | 97.9 |
| 5-6 | 5.25:1 | 42.5 | 81.5 | 1.77 | 98.4 | 97.3 |
| 5-7 | 5.25:1 | 42.5 | 84.3 | 1.30 | 98.9 | 98.0 |

### Example 6: Quench, Cleaning and Scrubbing of Outlet Gases

To reduce waste and handling of toxic reagents, the ATTM reaction has been developed using just enough ammonium sulfide to obtain a robust manufacturing process. Use of excess of reagent was avoided. It was initially observed that waste solutions from laboratory experiments developed a pressure which consisted at least in part of hydrogen sulfide, which meant that the waste solutions could not be sent off for incineration before quenching.

The excess ammonium sulfide (liberating toxic hydrogen sulfide) in mother liquors was quenched by addition of oxidants, sodium hypochlorite or hydrogen peroxide, and a H₂S detector was used to control the quenching.

Hydrogen peroxide (35% aqueous solution) was chosen as a primary oxidant of sulfur residues during quench in the reactors for safety reasons and ease of handling. Based on the isolated yield of ATTM expected for the process, the amount of hydrogen peroxide needed for the quench could be calculated. In theory, four equivalents of hydrogen peroxide are needed to fully oxidize hydrogen sulfide to the corresponding sulfate oxidation state. The excess unreacted ammonium sulfide was calculated, based on a conservative sample reaction yield of 83% and based on the fact that each molybdenum carries four sulfur atoms. Approximately 61 mole equivalents of hydrogen peroxide were thus needed to securely quench all of the excess hydrogen sulfide. Using this approach for calculation, the quench repeatedly produced a solution devoid of hydrogen sulfide and safe for disposal.

A safety concern connected to the quench is that it is highly exothermic, however, the exothermic reaction is addition controlled. When approximately 75% of the hydrogen peroxide had been added, the color of the quench solution changed, but more importantly, gas evolution started to increase. The gas evolution was also deemed addition controlled. The quench solution has a pH of approximately 10, and it was observed that the hydrogen peroxide addition increased the oxygen content in the reactor which is in line with an early report that states that hydrogen peroxide might liberate oxygen at alkaline pH. Dilution of the released oxygen by increased flushing with nitrogen was then used.

After the quench with hydrogen peroxide the quench solution was vented to remove dissolved gas before disposal. The most efficient method of cleaning the reactor in which the reaction was performed included an initial water wash to remove any residual peroxides, then top-filling with water to remove salts. After removal of the water, the reactor was rinsed twice with refluxing ethanol to remove sublimated residues in the condenser. Analysis of residues from the condenser confirmed that part of the isolated solids contained sulfur of an undetermined oxidation state.

A dilute basic solution of sodium hypochlorite in water was chosen as the scrubber media. This reagent is stable at basic pH which ensures higher solubility of hydrogen sulfide and also efficiently eliminated toxic sulfur gases.

### Example 7: Long-Term Stability Studies

ATTM Samples 1 and 2 were prepared essentially according to procedure in Example 1 starting with 12 kg of ammonium heptamolybdate. The isolated product was packed and stored immediately. Specifically, the samples were packaged corresponding to the packaging of the bulk material under argon in transparent anti-static polyethylene plastic bags, 90 10 10, 110 mm×170 mm, and the bags containing the samples were sealed with a plastic tie, and then subsequently packed in welded aluminium bags along with a drying agent in-between the two bags. Additional samples were packaged with ambient atmosphere in transparent anti-static polyethylene plastic bags, 90 10 10, 110 mm×170 mm. The bags containing the samples were sealed with a plastic tie, and then subsequently packed in welded aluminium bags along with a drying agent in-between the two bags. The results of the stability evaluation as determined by HPLC are presented below in Table 5, and show no significant degradation of ATTM after 6 months.

TABLE 5

| | Test conditions | | Sample 1 (w/w %) | | | Sample 2 (w/w %) | | |
|---|---|---|---|---|---|---|---|---|
| Impurity | Temp (° C.) | RH | Initial | 3 mo | 6 mo | Initial | 3 mo | 6 mo |
| ATTM | 5 | Air | 98.8 | 99.2 | 98.6 | 98.8 | 98.6 | 97.9 |
| | | Ar | 98.8 | 99.1 | 99.0 | 98.8 | 99.0 | 98.2 |
| Insoluble | | Air | <0.1 | n/a | <0.1 | <0.1 | n/a | <0.1 |
| materials | | Ar | <0.1 | n/a | <0.1 | <0.1 | n/a | <0.1 |
| ((NH₄)₆Mo₇O₂₄ | | Air | <0.05 | <0.05 | <0.05 | <0.05 | <0.05 | <0.05 |
| | | Ar | <0.05 | <0.05 | <0.05 | <0.05 | <0.05 | <0.05 |
| TM1 | | Air | <0.05 | <0.05 | <0.05 | <0.05 | <0.05 | <0.05 |
| | | Ar | <0.05 | <0.05 | <0.05 | <0.05 | <0.05 | <0.05 |
| TM2 | | Air | <0.05 | <0.05 | <0.05 | <0.05 | <0.05 | <0.05 |
| | | Ar | <0.05 | <0.05 | <0.05 | <0.05 | <0.05 | <0.05 |
| TM3 | | Air | 0.77 | 0.93 | 0.94 | 1.01 | 1.13 | 0.95 |
| | | Ar | 0.77 | 0.96 | 0.97 | 1.01 | 0.99 | 1.03 |
| Dimer S6 | | Air | <0.05 | <0.05 | <0.05 | <0.05 | <0.05 | <0.05 |
| | | Ar | <0.05 | <0.05 | <0.05 | <0.05 | <0.05 | <0.05 |
| ATTM | −20 | Air | 98.8 | 99.3 | 99.0 | 98.8 | 99.4 | 98.2 |
| | | Ar | 98.8 | 99.5 | 99.1 | 98.8 | 99.1 | 98.2 |
| Insoluble | | Air | <0.1 | n/a | <0.1 | <0.1 | n/a | <0.1 |
| materials | | Ar | <0.1 | n/a | <0.1 | <0.1 | n/a | <0.1 |
| ((NH₄)₆Mo₇O₂₄ | | Air | <0.05 | <0.05 | <0.05 | <0.05 | <0.05 | <0.05 |
| | | Ar | <0.05 | <0.05 | <0.05 | <0.05 | <0.05 | <0.05 |
| TM1 | | Air | <0.05 | <0.05 | <0.05 | <0.05 | <0.05 | <0.05 |
| | | Ar | <0.05 | <0.05 | <0.05 | <0.05 | <0.05 | <0.05 |
| TM2 | | Air | <0.05 | <0.05 | <0.05 | <0.05 | <0.05 | <0.05 |
| | | Ar | <0.05 | <0.05 | <0.05 | <0.05 | <0.05 | <0.05 |
| TM3 | | Air | 0.77 | 0.93 | 0.82 | 1.01 | 0.92 | 1.13 |
| | | Ar | 0.77 | 1.05 | 0.88 | 1.01 | 1.07 | 0.98 |
| Dimer S6 | | Air | <0.05 | <0.05 | <0.05 | <0.05 | <0.05 | <0.05 |
| | | Ar | <0.05 | <0.05 | <0.05 | <0.05 | <0.05 | <0.05 |

### Example 8: Process According to an Embodiment of the Disclosure

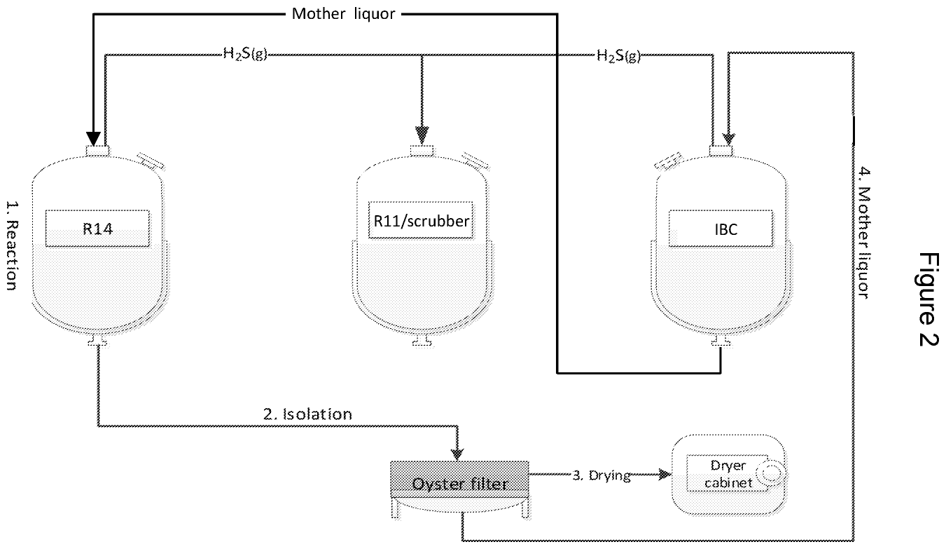

The reactor system used in this process is illustrated in FIG. 2. Briefly, the scrubber, reactor R11, containing a solution of sodium hypochlorite was connected to reactor R14 and to an intermediate bulk container (IBC). IBC was used for temporary storage of mother liquor. Ammonium heptamolybdate (13.3 kg) was added to reactor R14 followed by water (27 L) and ammonia (7.2 kg, 8 L) and stirred to form a solution. Ammonium sulfide (128 kg) was added over at least 30 minutes and the temperature was thereafter adjusted to 45±5° C. After 5 hours, ethanol (69 kg, 88 L) was added for at least 20 minutes at 45±5° C. The solution was then cooled down to 20±5° C. for at least 1 hour and then stirred for at least 12 hours. The product was isolated on a nutsche filter and the mother liquids were collected in an IBC container. The product on the nutsche filter was washed with a mixture of ethanol/water 2:1 (29 L), and the mother liquid and wash solution were transferred from the IBC container to R14. The filter-cake was washed further with ethanol (3×33 L). The wash liquid was transferred from the IBC container to R14. The product was collected and dried at 25° C. in a tray drier. The drying was done under reduced pressure to a constant weight. The product was thereafter packed under argon and stored in freezer.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Various exemplary embodiments of the disclosure include, but are not limited to the enumerated embodiments listed below, which can be combined in any number and in any combination that is not technically or logically inconsistent.

Embodiment 1 provides a process for manufacturing crystalline ammonium tetrathiomolybdate (ATTM), the process comprising:
contacting a molybdenum compound selected from ammonium heptamolybdate, ammonium dimolybdate, sodium molybdate, molybdenum oxide, and hydrate thereof with water and ammonia in a reaction vessel under stirring to obtain a molybdenum compound solution;
providing ammonium sulfide to the molybdenum compound solution in an amount corresponding to a S:Mo molar ratio in a range of 4.5:1 to 6.5:1 over a period of time sufficient to obtain a reaction mixture;
maintaining the reaction mixture at a temperature in a range of 35° C. to 55° C. for a reaction time of at least 4 hours to create a slurry;
optionally providing a crystallization solvent to the slurry;
optionally maintaining the slurry at a temperature in a range of 10° C. to 30° C. for at least 2 hours; and
removing liquid from the slurry to obtain crystalline ATTM.

Embodiment 2 provides the process of embodiment 1, wherein the molybdenum compound is selected from ammonium heptamolybdate, ammonium dimolybdate, sodium molybdate, and hydrate thereof.

Embodiment 3 provides the process of embodiment 1, wherein the molybdenum compound is ammonium heptamolybdate or ammonium heptamolybdate tetrahydrate; or wherein the molybdenum compound is ammonium heptamolybdate; or wherein the molybdenum compound is ammonium heptamolybdate tetrahydrate.

Embodiment 4 provides the process of embodiment 1, wherein the molybdenum compound is ammonium dimolybdate.

Embodiment 5 provides the process of any one of embodiments 1-4, wherein ammonium sulfide is provided in an amount corresponding to a S:Mo molar ratio in a range of 4.5:1 to 6.2:1; or 4.5:1 to 6:1; or 4.5:1 to 5.8:1; or 4.5:1 to 5.5:1; or 4.5:1 to 5.3:1; or 4.5:1 to 5:1; or 4.8:1 to 6.5:1; or 4.8:1 to 6.2:1; or 4.8:1 to 6:1; or 4.8:1 to 5.8:1; or 4.8:1 to 5.5:1; or 4.8:1 to 5.3:1; or 4.8:1 to 5:1; or 5:1 to 6.5:1; or 5:1 to 6.2:1; or 5:1 to 6:1; or 5:1 to 5.8:1; or 5:1 to 5.5:1; or 5:1 to 5.3:1; or 5.2:1 to 6.5:1; or 5.2:1 to 6.2:1; or 5.2:1 to 6:1; or 5.2:1 to 5.8:1; or 5.2:1 to 5.5:1; or 5.5:1 to 6.5:1; or 5.5:1 to 6.2:1; or 5.5:1 to 6:1; or 5.5:1 to 5.8:1; or 5.3:1 to 5.7:1; or 5.4:1 to 5.6:1; or 5.45:1 to 5.55:1.

Embodiment 6 provides the process of any one of embodiments 1-4, wherein ammonium sulfide is provided in an amount corresponding to a S:Mo molar ratio of about 5.5:1; or about 6:1; about 5:1; about 4.5:1.

Embodiment 7 provides the process of any one of embodiments 1-6, wherein the period of time sufficient to obtain a reaction mixture is at least 15 minutes, or at least 20 minutes, or at least 30 minutes.

Embodiment 8 provides the process of any one of embodiments 1-7, wherein the reaction mixture is maintained at a temperature in a range of 40° C. to 55° C.; or 45° C. to 55° C.; or 50° C. to 55° C.; or 35° C. to 50° C.; or 40° C. to 50° C.; or 45° C. to 50° C.; or 35° C. to 45° C.; or 40° C. to 45° C.; or 45° C. to 50° C.; or 37° C. to 53° C.; or 38° C. to 52° C.; or 41° C. to 49° C.; or 42° C. to 48° C.; or 43° C. to 47° C.; or 44° C. to 46° C.

Embodiment 9 provides the process of any one of embodiments 1-7, wherein the reaction mixture is maintained at a temperature of about 45° C.

Embodiment 10 provides the process of any one of embodiments 1-9, wherein the reaction time is at least 4.5 hours; or at least 5 hours; or in a range of 4 hours to 8 hours; or in a range of 4 hours to 7 hours; or in a range of 4 hours to 6 hours; or in a range of 4 hours to 5 hours; or in a range of 4.5 hours to 8 hours; or in a range of 4.5 hours to 7 hours; or in a range of 4.5 hours to 6 hours; or in a range of 4.5 hours to 5 hours; or in a range of 5 hours to 8 hours; or in a range of 5 hours to 7 hours; or in a range of 5 hours to 6 hours; or in a range of 5.5 hours to 6 hours; or in a range of 4.5 hours to 5.5 hours; or in a range of 4.6 hours to 5.4 hours; or in a range of 4.7 hours to 5.3 hours; or in a range of 4.8 hours to 5.2 hours; or in a range of 4.9 hours to 5.1 hours.

Embodiment 11 provides the process of any one of embodiments 1-9, wherein the reaction time is about 5 hours.

Embodiment 12 provides the process of any one of embodiments 1-11, wherein the crystallization solvent is provided to the slurry.

Embodiment 13 provides the process of embodiment 12, wherein the crystallization solvent is selected from methanol, ethanol, ethylene glycol, petroleum ether, n-hexane, tetrahydrofuran, toluene, water, and mixtures thereof; or wherein the crystallization solvent is ethanol.

Embodiment 14 provides the process of embodiment 12 or 13, wherein the solvent is provided for at least 15 minutes; or for at least 20 minutes; or for at least 25 minutes; or over a time in the range of 15 minutes to 30 minutes; or 15 minutes to 25 minutes; or 15 minutes to 20 minutes; or 20 minutes to 30 minutes; or 20 minutes to 25 minutes; or 18 minutes to 22 minutes; or for about 20 minutes.

Embodiment 15 provides the process of any one of embodiments 12-14, wherein the solvent is provided for at a temperature in a range of 40° C. to 55° C.; or 45° C. to 55° C.; or 50° C. to 55° C.; or 35° C. to 50° C.; or 40° C. to 50° C.; or 45° C. to 50° C.; or 35° C. to 45° C.; or 40° C. to 45° C.; or 45° C. to 50° C.; or 37° C. to 53° C.; or 38° C. to 52° C.; or 41° C. to 49° C.; or 42° C. to 48° C.; or 43° C. to 47° C.; or 44° C. to 46° C.

Embodiment 16 provides the process of any one of embodiments 12-14, wherein the solvent is provided for at a temperature of about 45° C.

Embodiment 17 provides the process of any one of embodiments 1-16, wherein the slurry is maintained at a temperature in a range of 10° C. to 30° C. for at least 3 hours; or at least 5 hours; or at least 6 hours; or at least 7 hours; or at least 8 hours; or at least 9 hours; or at least 10 hours; or at least 11 hours; or at least 12 hours; or for a time in the range of 8 hours to 16 hours; or 10 hours to 16 hours; or 8 hours to 14 hours; or 10 hours to 14 hours; or 8 hours to 12 hours; or 10 hours to 12 hours.

Embodiment 18 provides the process of any one of embodiments 1-17, wherein the slurry is maintained at a temperature in a range of 10° C. to 25° C.; or 10° C. to 20° C.; or 10° C. to 15° C.; or 15° C. to 30° C.; or 15° C. to 25° C.; or 15° C. to 20° C.; or 20° C. to 30° C.; or 18° C. to 22° C.; or 19° C. to 21° C.; or at a temperature of about 15° C.; or about 20° C.; or about 25° C.

Embodiment 19 provides the process of any one of embodiments 1-18, further comprising gradually cooling the slurry to the temperature in a range of 10° C. to 30° C. prior to maintaining at a cooling rate of 0.2-1° C./minute; or about 0.6° C./minute; or about 0.5° C./min.

Embodiment 20 provides the process of embodiment 19, wherein gradually cooling is over a period of at least 30 minutes; or at least 45 minutes; or at least 1 hour; or between 30 minutes and 1 hour.

Embodiment 21 provides the process of any one of embodiments 1-20, wherein the liquid is removed from the slurry by filtration or centrifugation.

Embodiment 22 provides the process of embodiment 21, wherein filtration is by nutsche filter, Buechner filter, sintered glass filter, or paper filter.

Embodiment 23 provides the process of any one of embodiments 1-22 further comprising washing the crystalline ATTM with at least one of an alcohol, particularly ethanol, water, or ethanol:water mixture.

Embodiment 24 provides the process of any one of embodiments 1-22 further comprising washing the crystalline ATTM with a 2:1 ethanol:water.

Embodiment 25 provides the process of any one of embodiments 1-24 further comprising washing the crystalline ATTM with an alcohol, particularly ethanol.

Embodiment 26 provides the process of any one of embodiments 1-25 further comprising drying the crystalline ATTM at about 25° C. under reduced pressure (e.g. in a vacuum oven).

Embodiment 27 provides the process of any one of embodiments 1-26 further comprising storing the crystalline ATTM under argon.

Embodiment 28 provides the process of embodiment 27, wherein storing is at a reduced temperature (e.g., from −20° C. to 0° C.; or about −15° C.; of about −18° C.; or about −20° C.).

Embodiment 29 provides the process of any one of embodiments 1-28, wherein hydrogen sulfide is obtained as a byproduct, and is further vented into a second reaction vessel comprising a sodium hypochlorite or hydrogen peroxide solution.

Embodiment 30 provides the process of any one of embodiments 1-29, wherein the molybdenum compound is provided in an amount of at least 1 kg; or at least 2 kg; or at least 5 kg; or at least 10 kg.

Embodiment 31 provides the process of any one of embodiments 1-30, wherein crystalline ATTM is obtained in a yield of at least 70%, or at least 75%; or at least 80%.

Embodiment 32 provides the process of any one of embodiments 1-31 configured to comply with good manufacturing process (cGMP) standard.

Embodiment 33 provides the process of any one of embodiments 1-32, wherein crystalline ATTM is at least 88%, or at least 89%, or at least 90%, or at least 91%, or at least 92%, or at least 93%, or at least 94%, or at least 95%, or at least 96%, or at least 97%, or at least 98%, or at least 99%, or at least 100% (w/w) pure.

Embodiment 34 provides the process of any one of embodiments 1-33, wherein crystalline ATTM comprises less than 9%, or less than 8%, or less than 7%, or less than 6%, or less than 5%, or less than 4%, or less than 3%, or less than 2.5%, or less than 2%, or less than 1%, or about 2% (w/w) of [MoOS₃]²⁻ impurity.

Embodiment 35 provides the process of any one of embodiments 1-34, wherein crystalline ATTM comprises less than 10%, or less than 9%, or less than 8%, or less than 7%, or less than 6%, or less than 5%, or less than 4% (w/w) of impurities selected from one or more of [MoO₄]²⁻, [MoO₃S]²⁻, [MoO₂S₂]²⁻, and [MoOS₃]²⁻.

Embodiment 36 provides the process of any one of embodiments 1-35, wherein crystalline ATTM comprises no more than 1%, or no more than 0.8%, or no more than 0.6%, or no more than 0.5%, or no more than 0.1%, or no more than 0.05%, or no more than 0.01% (w/w) of polymeric molybdenum impurities.

Embodiment 37. A crystalline ammonium tetrathiomolybdate (ATTM) having a pharmaceutical grade purity and obtained by process of any one of embodiments 1-32.

Embodiment 38 provides the crystalline ATTM of embodiment 37, which is at least 88%, or at least 89%, or at least 90%, or at least 91%, or at least 92%, or at least 93%, or at least 94%, or at least 95%, or at least 96%, or at least 97%, or at least 98%, or at least 99%, or at least 100% (w/w) pure.

Embodiment 39 provides the crystalline ATTM of embodiment 37 or 38, comprising less than 9%, or less than 8%, or less than 7%, or less than 6%, or less than 5%, or less than 4%, or less than 3%, or less than 2.5%, or less than 2%, or less than 1%, or about 2% (w/w) of [MoOS₃]²⁻ impurity.

Embodiment 40 provides the crystalline ATTM of any one of embodiments 37-39, comprising less than 10%, or less than 9%, or less than 8%, or less than 7%, or less than 6%, or less than 5%, or less than 4% (w/w) of total molybdenum impurities, wherein the molybdenum impurities are selected from one or more of [MoO₄]²⁻, [MoO₃S]²⁻, [MoO₂S₂]²⁻, and [MoOS₃]²⁻.

Embodiment 41 provides the crystalline ATTM of any one of embodiments 37-40, comprising no more than 1%, or no more than 0.8%, or no more than 0.6%, or no more than 0.5%, or no more than 0.1%, or no more than 0.05%, or no more than 0.01% (w/w) of polymeric molybdenum impurities.

Embodiment 42 provides a process for manufacturing bis-choline tetrathiomolybdate, wherein bis-choline tetrathiomolybdate has a pharmaceutical grade purity, the process comprising:
contacting an aqueous choline hydroxide solution with a crystalline ammonium tetrathiomolybdate according to any one of embodiments 37-41 and water to obtain a reaction mixture;
maintaining the reaction mixture at a temperature in a range of 10° C. to 40° C. for a reaction time sufficient to obtain crude bis-choline tetrathiomolybdate; and
providing ethanol to the reaction mixture at a temperature in a range of 35° C. to 55° C. over a period of time sufficient to obtain bis-choline tetrathiomolybdate.

Embodiment 43 provides the process of embodiment 42, wherein the bis-choline tetrathiomolybdate obtained comprises less than 0.1%, or less than 0.09%, or less than 0.05% (w/w) of [MoOS₃]²⁻ impurity.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A process for manufacturing crystalline ammonium tetrathiomolybdate (ATTM), the process comprising:
contacting a molybdenum compound selected from ammonium heptamolybdate, ammonium dimolybdate, sodium molybdate, molybdenum oxide, and hydrate thereof with water and ammonia in a reaction vessel under stirring to obtain a molybdenum compound solution;
providing ammonium sulfide to the molybdenum compound solution in an amount corresponding to a S:Mo molar ratio in a range of 4.5:1 to 6.5:1 over a period of time sufficient to obtain a reaction mixture;
maintaining the reaction mixture at a temperature in a range of 35° C. to 55° C. for a reaction time of at least 4 hours to create a slurry;
optionally providing a crystallization solvent to the slurry;
optionally maintaining the slurry at a temperature in a range of 10° C. to 30° C. for at least 2 hours; and
removing liquid from the slurry to obtain crystalline ATTM.

2. The process of claim 1, wherein the molybdenum compound is selected from ammonium heptamolybdate, ammonium dimolybdate, sodium molybdate, and hydrate thereof.

3. The process of claim 1, wherein the molybdenum compound is ammonium heptamolybdate or ammonium heptamolybdate tetrahydrate.

4. The process of claim 1, wherein the molybdenum compound is ammonium dimolybdate.

5. The process of any one of claims 1-4, wherein ammonium sulfide is provided in an amount corresponding to a S:Mo molar ratio in a range of 4.5:1 to 6.2:1.

6. The process of any one of claims 1-5, wherein the period of time sufficient to obtain a reaction mixture is at least 15 minutes.

7. The process of any one of claims 1-6, wherein the reaction mixture is maintained at a temperature in a range of 40° C. to 55° C.

8. The process of any one of claims 1-7, wherein the reaction time is in a range of 4 hours to 8 hours.

9. The process of any one of claims 1-11, wherein the crystallization solvent is provided to the slurry, and wherein the crystallization solvent is selected from methanol, ethanol, ethylene glycol, petroleum ether, n-hexane, tetrahydrofuran, toluene, water, and mixtures thereof.

10. The process of claim 9, wherein the solvent is provided over a time in the range of 15 minutes to 30 minutes.

11. The process of any one of claims 1-10, wherein the slurry is maintained at a temperature in a range of 10° C. to 30° C. for at least 3 hours.

12. The process of any one of claims 1-11, further comprising gradually cooling the slurry to the temperature in a range of 10° C. to 30° C. prior to maintaining at a cooling rate of 0.2-1° C./minute.

13. The process of any one of claims 1-12, further comprising washing the crystalline ATTM with at least one of an alcohol, water, or a mixture thereof.

14. The process of any one of claims 1-12, further comprising washing the crystalline ATTM with a 2:1 ethanol:water or with ethanol.

15. The process of any one of claims 1-14, wherein hydrogen sulfide is obtained as a byproduct, and is further vented into a second reaction vessel comprising a sodium hypochlorite or hydrogen peroxide solution.

16. A crystalline ammonium tetrathiomolybdate (ATTM) having a pharmaceutical grade purity and obtained by the process of any one of claims 1-15.

17. The crystalline ATTM of claim 16, comprising less than 9% (w/w) of [MoOS₃]²⁻ impurity.

18. The crystalline ATTM of claim 16 or 17, comprising less than 10% (w/w) of total molybdenum impurities, wherein the molybdenum impurities are selected from one or more of [MoO₄]²⁻, [MoO₃S]²⁻, [MoO₂S₂]²⁻, and [MoOS₃]²⁻.

19. A process for manufacturing bis-choline tetrathiomolybdate, wherein bis-choline tetrathiomolybdate has a pharmaceutical grade purity, the process comprising:
contacting an aqueous choline hydroxide solution with a crystalline ammonium tetrathiomolybdate according to any one of claims 16-18 and water to obtain a reaction mixture;
maintaining the reaction mixture at a temperature in a range of 10° C. to 40° C. for a reaction time sufficient to obtain crude bis-choline tetrathiomolybdate; and
providing ethanol to the reaction mixture at a temperature in a range of 35° C. to 55° C. over a period of time sufficient to obtain bis-choline tetrathiomolybdate.

20. The process of claim 19, wherein the bis-choline tetrathiomolybdate obtained comprises less than 0.1% (w/w) of [MoOS₃]²⁻ impurity.
